SEQUENCE LISTING (10 sequences)

SEQ ID NO: 1 - DNA, 2681 bp, Homo Sapiens
gaggagagagagagagtaaggagccagccatgaatcctttccagaaaaatgagtccaaggaaactcttttttcacctgtctccattgaagaggtaccacctcgaccacctagccctccaaagaagccatctccgacaatctgtggctccaactatccactgagcattgccttcattgtggtgaatgaattctgcgagcgcttttcctattatggaatgaaagctgtgctgatcctgtatttcctgtatttcctgcactggaatgaagatacctccacatctatataccatgccttcagcagcctctgttattttactcccatcctgggagcagccattgctgactcgtggttgggaaaattcaagacaatcatctatctctccttggtgtatgtgcttggccatgtgatcaagtccttgggtgccttaccaatactgggaggacaagtggtacacacagtcctatcattgatcggcctgagtctaatagctttggggacaggaggcatcaaaccctgtgtggcagcttttggtggagaccagtttgaagaaaaacatgcagaggaacggactagatacttctcagtcttctacctgtccatcaatgcagggagcttgatttctacatttatcacacccatgctgagaggagatgtgcaatgttttggagaagactgctatgcattggcttttggagttccaggactgctcatggtaattgcacttgttgtgtttgcaatgggaagcaaaatatacaataaaccaccccctgaaggaaacatagtggctcaagttttcaaatgtatctggtttgctatttccaatcgtttcaagaaccgttctggagacattccaaagcgacagcactggctagactgggcagctgagaaatatccaaagcagctcattatggatgtaaaggcactgaccagggtactattcctttatatcccattgcccatgttctgggctcttttggatcagcagggttcacgatggactttgcaagccatcaggatgaataggaatttggggttttttgtgcttcagccggaccagatgcaggttctaaatccctttctggttcttatcttcatcccgttgtttgactttgtcatttatcgtctggtctccaagtgtggaattaacttctcatcacttaggaaaatggctgttggtatgatcctagcgtgcctggcatttgcagttgcggcagctgtagagataaaaataaatgaaatggccccagcccagtcaggtccccaggaggttttcctacaagtcttgaatctggcagatgatgaggtgaaggtgacagtggtgggaaatgaaaacaattctctgttgatagagtccatcaaatcctttcagaaaacaccacactattccaaactgcacctgaaaacaaaaagccaggattttcacttccacctgaaatatcacaatttgtctctctacactgagcattctgtgcaggagaagaactggtacagtcttgtcattcgtgaagatgggaacagtatctccagcatgatggtaaaggatacagaaagcaaaacaaccaatgggatgacaaccgtgaggtttgttaacactttgcataaagatgtcaacatctccctgagtacagatacctctctcaatgttggtgaagactatggtgtgtctgcttatagaactgtgcaaagaggagaataccctgcagtgcactgtagaacagaagataagaacttttctctgaatttgggtcttctagactttggtgcagcatatctgtttgttattactaataacaccaatcagggtcttcaggcctggaagattgaagacattccagccaacaaaatgtccattgcgtggcagctaccacaatatgccctggttacagctggggaggtcatgttctctgtcacaggtcttgagttttcttattctcaggctccctctagcatgaaatctgtgctccaggcagcttggctattgacaattgcagttgggaatatcatcgtgcttgttgtggcacagttcagtggcctggtacagtgggccgaattcattttgttttcctgcctcctgctggtgatctgcctgatcttctccatcatgggctactactatgttcctgtaaagacagaggatatgcggggtccagcagataagcacattcctcacatccaggggaacatgatcaaactagagaccaagaagacaaaactctgatgacttcctagattctgtcctgaccccaattcctggccctgtcttgaagcattttttttcttctactggattagacaagagagatagcagcatatcagagctgatctcctccacctttctccaatgacagaagttccaggactggttttccagtacatctttaaacaaggccccagagactctatgtctgcccgtccatcagtgaactcattaaaacttgtgcagtgttgctggagctggcctggtgtctccaaatgaccatgaaaatacacacgtataatggagatcattctctgtgggtatgcaaagttatgggaattcctttataggtaactgccatttaggactgatggccctaatttttgaggtgctgatttagaggcaaaattgcagaataacaaagaaatggtatttcaagtttttttttttataagcaatgtaattatgctattcacaggggcc

SEQ ID NO: 2 - protein, 729 aa, Homo Sapiens [sequence omitted]

SEQ ID NO: 3 - DNA, 363 bp, Homo Sapiens
gaggtccagctgcaacagtctggacctgagctggtgaagcctggagcttcaatgaagatatcctgcaaggcttctggttactcattcactggctacaccatgaactgggtgaagcagagccatggaaagaaccttgagtggattggacttattaatccttacaatggtggtattaactacaaccagaagttcaagggcaaggccacattaactgtagacaagtcatccagtacagcctacatggagctcctcagtctgacatctgaggactctgcagtctattactgtacaagacgggcctactatggtaactacggtactatggactactggggtcaaggaacctcagtcaccgtctcctca

SEQ ID NO: 4 - DNA, 321 bp, Homo Sapiens
gaaaatgttctcacccagtctccagcaagcatgtctgcatctccaggggaaaaggtcaccatgacctgcagtgccagctcaagtgtaagttacatgcactggtaccagcagaagtcaaccacctcccccaaactctggatttatgacacatccaatctggcttctggggtcccaggtcgcttcagtggcagtgggtctggaaactcttactctctcacgatcagcaacatggaggctgaagatgttgccacttattactgttttcaggggagtggttacccactcacgttcggtgctgggaccaagctggagctgaaacgg

SEQ ID NO: 5 - DNA, 354 bp, Homo Sapiens
caggtccaactgcagcagcctggggctgagctggtgaggcctggggcttcagtgaagctgtcctgcaaggcttctggctacaccttcaccagctactggttgaactgggtgaggcagaggcctggacaaggccttgaatggattggtatgattgatccttcagacagtgaaactcactacaatcaaatgttcaaggacaaggccacattgactgtagacaagtcctccagcacagcctacatgcagctcagcagcctgacatctgaggactctgcggtctattactgtacaagtcagggggtaccggtcccctttgactactggggccaaggcaccactctcacagtctcctca

SEQ ID NO: 6 - DNA, 339 bp, Homo Sapiens
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacagagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaacgg

SEQ ID NO: 7 - protein, 121 aa, Homo Sapiens
EVQLQQSGPELVKPGASMKISCKASGYSFTGYTMNWVKQSHGKNLEWIGLINPYNGGINYNQKFKGKATLTVDKSSSTAYMELLSLTSEDSAVYYCTRRAYYGNYGTMDYWGQGTSVTVSS

SEQ ID NO: 8 - protein, 107 aa, Homo Sapiens
ENVLTQSPASMSASPGEKVTMTCSASSSVSYMHWYQQKSTTSPKLWIYDTSNLASGVPGRFSGSGSGNSYSLTISNMEAEDVATYYCFQGSGYPLTFGAGTKLELKR

SEQ ID NO: 9 - protein, 118 aa, Homo Sapiens
QVQLQQPGAELVRPGASVKLSCKASGYTFTSYWLNWVRQRPGQGLEWIGMIDPSDSETHYNQMFKDKATLTVDKSSSTAYMQLSSLTSEDSAVYYCTSQGVPVPFDYWGQGTTLTVSS

SEQ ID NO: 10 - protein, 113 aa, Homo Sapiens
DVVMTQTPLSLPVSLGDQASISCRSSQSLVHSNGNTYLHWYLQKPGQSPKLLIYRVSNRFSGVPDRFSGSGSGTDFTLKISRVEAEDLGVYFCSQSTHVPWTFGGGTKLEIKR

(19) United States
(12) Patent Application Publication
(10) Pub. No.: US 2004/0223970 A1
(43) Pub. Date: Nov. 11, 2004

(54) Antibodies against SLC15A2 and uses thereof

(76) Inventors: Daniel Afar, Fremont, CA (US); Debbie Law, San Francisco, CA (US)

Correspondence Address: HOWREY SIMON ARNOLD & WHITE, LLP, 301 Ravenswood Avenue, Box No. 34, Menlo Park, CA, 94025, US

(21) Appl. No.: 10/789,090

(22) Filed: Feb. 27, 2004

Related U.S. Application Data
(60) Provisional application No. 60/451,294, filed on Feb. 28, 2003.

(51) Int. Cl. A61K 39/395; C07K 16/30

(52) U.S. Cl. 424/155.1; 530/388.8

(57) ABSTRACT

The invention relates to the identification and generation of antibodies that specifically bind to SLC15A2 protein. The disclosed anti-SLC15A2 antibodies and compositions comprising them may be used for diagnosis, prognosis and therapy of cancer (including metastatic cancer) or fibrotic conditions, e.g., ovarian cancer, cervical cancer, prostate cancer, uterine cancer, lung cancer, lung fibrosis, and glioblastoma. The disclosed antibodies include conjugates with cytotoxic effector components that are useful for cancer therapy.

### CROSS-REFERENCES TO RELATED APPLICATIONS

This application claims priority from U.S. Provisional Application No. 60/451,294 filed Feb. 28, 2003, which is hereby incorporated by reference herein in its entirety.

This application also is related to U.S. Ser. No. 10/245,882 filed Sep. 17, 2002; and U.S. Ser. No. 10/295,027 filed Nov. 13, 2002; each of which is hereby incorporated by reference in its entirety.

### FIELD OF THE INVENTION

The invention relates to the identification and generation of antibodies that specifically bind to SLC15A2 proteins; and to the use of such antibodies and compositions comprising them in the diagnosis, prognosis and therapy of cancer.

### BACKGROUND OF THE INVENTION

The SLC15A2 (Solute carrier family 15 (H+/peptide transporter), member 2; LocusLink 6565, OMIM 602339) protein has been implicated in certain cancerous or fibrotic conditions, e.g., ovarian cancer, cervical cancer, prostate cancer, uterine cancer, lung cancer, lung fibrosis, and glioblastoma. Antibodies useful for diagnosis, prognosis, and effective treatment of cancer, including metastatic cancer, would be desirable. Accordingly, provided herein are compositions and methods that can be used in diagnosis, prognosis, and therapy of such conditions.

### SUMMARY OF THE INVENTION

The present invention provides anti-SLC15A2 antibodies that are useful for making conjugated antibodies for therapeutic purposes. For example, the anti-SLC15A2 antibodies of the invention are useful as selective cytotoxic agents for SLC15A2 expressing cells. In some embodiments, the antibodies of the present invention are therapeutically useful in persons diagnosed with cancer and other proliferative conditions, including benign proliferative conditions. In one aspect, the antibodies of the present invention can be used to treat proliferative conditions of the ovary including, for example, ovarian cancer.

The present invention provides antibodies that competitively inhibit binding of proteins encoded by vectors containing some or all of the sequence associated with SLC15A2 (Hs.118747; see GenBank entries NM_{—}021082.2 and XM_{—}002922.3; see U.S. Ser. No. 10/245,882). In some embodiments the antibodies are further conjugated to an effector component. The effector component can be a label (e.g., a fluorescent label, an effector domain, e.g., MicA) or can be a cytotoxic moiety (e.g., a radioisotope or a cytotoxic chemical) An exemplary cytotoxic chemical is auristatin. In other embodiments the antibodies can be used alone to inhibit tumor cell growth.

The antibodies of the invention can be whole antibodies or can be antibody fragments. In some embodiments the immunoglobulin is a humanized antibody. An exemplary antibody of the invention is defined by CDRs.

The invention also provides pharmaceutical compositions comprising a pharmaceutically acceptable excipient and the antibody of the invention. In these embodiments, the antibody can be further conjugated to an effector component. The effector component can be a label (e.g., a fluorescent label) or can be cytotoxic moiety (e.g., a radioisotope or a cytotoxic chemical) An exemplary cytotoxic chemical is auristatin. The antibodies in the pharmaceutical compositions can be whole antibodies or can antibody fragments. In some embodiments the immunoglobulin is a humanized antibody.

The invention further provides immunoassays using the immunoglobulins of the invention. These methods involve detecting a cancer cell in a biological sample from a patient by contacting the biological sample with an antibody of the invention. The antibody is typically conjugated to a label such as fluorescent or other label.

The invention provides methods of inhibiting proliferation of a cancer- or fibrosis-associated cell. The method comprises contacting the cell with an antibody of the invention. In most embodiments, the cancer cell is in a patient, typically a human. The patient may be undergoing a therapeutic regimen to treat metastatic ovarian cancer or may be suspected of having ovarian cancer.

Thus, in one aspect the invention provides an antibody whose binding to SLC15A2 is competitively inhibited by the presence of a second antibody comprising a CDR of PDO5 #810 or 811.

In one aspect, the antibody whose binding to SLC15A2 is competitively inhibited by the presence of a second antibody is further conjugated to an effector component. In one aspect, the effector component is a fluorescent label. In another aspect, the effector component is a radioisotope or a cytotoxic chemical. In another aspect the cytotoxic chemical is auristatin.

In one aspect, the antibody whose binding to SLC15A2 is competitively inhibited by the presence of a second antibody comprising a CDR of PDO5 #810 or 811 is an antibody fragment. In another aspect the antibody is a humanized antibody. In another aspect the antibody whose binding to SLC15A2 is competitively inhibited by the presence of a second antibody comprising a CDR of PDO5 #810 or 811 is PDO5 #810 or #811.

In still another aspect, the SLC15A2 that is bound by an antibody whose binding is competitively inhibited by the presence of a second antibody comprising a CDR of PDO5 #810 or 811, is on a cancer or fibrosis cell.

In another aspect the invention provides a pharmaceutical composition comprising a pharmaceutically acceptable excipient and an antibody whose binding to SLC15A2 is competitively inhibited by the presence of a second antibody comprising a CDR of PDO5 #810 or 811. In one aspect the antibody contained in the pharmaceutical composition is further conjugated to an effector component. In one aspect the effector component is a fluorescent label. In another aspect, the effector component is a radioisotope or a cytotoxic chemical moiety. In another aspect, the cytotoxic chemical is auristatin. In another aspect the antibody contained in the pharmaceutical composition is a humanized antibody. In still another aspect the antibody contained in the pharmaceutical composition is PDO5.

The invention also provides a method of detecting an ovarian cancer, uterine cancer, prostate cancer, lung cancer, glioblastoma, cervical cancer or fibrosis cell in a biological sample from a patient, the method comprising contacting the biological sample with an antibody whose binding to SLC15A2 is competitively inhibited by the presence of a second antibody comprising a CDR of PDO5 #810 or 811. In one aspect the antibody used in the method is further conjugated to a fluorescent label.

The invention further provides a method of inhibiting proliferation of an ovarian, prostate, lung, uterine, brain, cervical or fibrosis-associated cell, the method comprising the step of contacting the cell with an antibody whose binding to SLC15A2 is competitively inhibited by the presence of a second antibody comprising a CDR of PDO5 #810 or 811. In one aspect the method employs an antibody fragment.

The invention also provides an antibody comprising SEQ ID NO: 7-10 or a CDR sequence therefrom. In one aspect, the antibody comprising SEQ ID NO: 7-10 or a CDR sequence therefrom binds to SLC15A2; or is further conjugated to an effector compound.

In another aspect the invention provides a pharmaceutical composition comprising a pharmaceutically acceptable excipient and an antibody comprising SEQ ID NO: 7-10 or a CDR therefrom which binds to SLC15A2; and which may also be further conjugated to an effector compound.

The invention also provides a method of detecting a cancer or fibrosis cell in a biological sample from a patient, the method comprising contacting the biological sample with an antibody comprising SEQ ID NO: 7-10 or a CDR therefrom that binds to SLC15A2; and may be further conjugated to an effector compound.

Finally the invention provides a method of inhibiting proliferation of an ovarian, prostate, lung, or cervical cancer or fibrosis-associated cell, the method comprising the step of contacting the cell with an antibody comprising SEQ ID NO: 7-10 or a CDR therefrom that binds to SLC15A2; and that may be further conjugated to an effector compound.

### BRIEF DESCRIPTION OF THE DRAWINGS

FIG. 1 depicts data from FACS analysis of monoclonal antibodies PDO5 #802, #807, #810, and #811.

FIG. 2 depicts effect of PDO5 monoclonal antibodies ligated with a secondary antibody crosslinked to saporin.

FIG. 3 depicts a plot of SLC15A2 gene expression level versus tissue type illustrating that this gene is up-regulated in prostate cancer.

FIG. 4 depicts staining of human prostate tissue sections with monoclonal antibody PDO5 #810.

### DETAILED DESCRIPTION OF THE INVENTION

The present invention provides novel reagents and methods for treatment, diagnosis and prognosis for certain cancers using antibodies against SLC15A2. Some of the conditions detectable and treatable with SLC15A2 antibodies include, but are not limited to, prostate cancer, lung cancer, uterine cancer, ovarian cancer, cervical cancer, lung fibrosis, and glioblastoma. In particular, the present invention provides anti-SLC15A2 antibodies that are particularly useful as selective cytotoxic agents for SLC15A2 expressing cells.

Epitope mapping of antibodies showing high affinity binding can be carried out through competitive binding analyses. Using this methodology antibodies recognizing a number of individual epitopes can be identified or distinguished. The antibodies are then assessed for SLC15A2 dependent cell death in vitro. Using these methods antibodies that promote significant cell death can be identified.

### Definitions

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′)₂, Fab, Fv and rIgG. See also, Pierce Catalog and Handbook, 1994-1995 (Pierce Chemical Co., Rockford, Ill.). See also, e.g., Kuby, J., Immunology, 3^{rd} Ed., W. H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol:5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

An antibody immunologically reactive with a particular antigen can be generated by recombinant methods such as selection of libraries of recombinant antibodies in phage or similar vectors, see, e.g., Huse et al., Science 246:1275-1281 (1989); Ward et al., Nature 341:544-546 (1989); and Vaughan et al., Nature Biotech. 14:309-314 (1996), or by immunizing an animal with the antigen or with DNA encoding the antigen.

Typically, an immunoglobulin has a heavy and light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs”. The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

References to “V_{H}” or a “VH” refer to the variable region of an immunoglobulin heavy chain of an antibody, including the heavy chain of an Fv, scFv , or Fab. References to “V_{L}” or a “VL” refer to the variable region of an immunoglobulin light chain, including the light chain of an Fv, scFv , dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The term “SLC15A2 protein” or “SLC15A2 polynucleotide” refers to nucleic acid and polypeptide polymorphic variants, alleles, mutants, and interspecies homologues that: (1) have a nucleotide sequence that has greater than about 60% nucleotide sequence identity, 65%, 70%, 75%, 80%, 85%, 90%, preferably 91%, 92%, 93%, 94%, 95%, 96%, 97%, 98% or 99% or greater nucleotide sequence identity, preferably over a region of over a region of at least about 25, 50, 100, 200, 500, 1000, or more nucleotides, to a nucleotide sequence of SEQ ID NO:1; (2) bind to antibodies, e.g., polyclonal antibodies, raised against an immunogen comprising an amino acid sequence encoded by a nucleotide sequence of SEQ ID NO: 1, and conservatively modified variants thereof; (3) specifically hybridize under stringent hybridization conditions to a nucleic acid sequence, or the complement thereof of SEQ ID NO: 1 and conservatively modified variants thereof or (4) have an amino acid sequence that has greater than about 60% amino acid sequence identity, 65%, 70%, 75%, 80%, 85%, 90%, preferably 91%, 92%, 93%, 94%, 95%, 96%, 97%, 98%, or 99% or greater amino sequence identity, preferably over a region of at least about 25, 50, 100, 200, or more amino acids, to an amino acid sequence of SEQ ID NO:2. A polynucleotide or polypeptide sequence is typically from a mammal including, but not limited to, primate, e.g., human; rodent, e.g., rat, mouse, hamster; cow, pig, horse, sheep, or other mammal. An “SLC15A2 polypeptide” and an “SLC15A2 polynucleotide,” include both naturally occurring or recombinant forms.

A “full length” SLC15A2 protein or nucleic acid refers to a ovarian cancer polypeptide or polynucleotide sequence, or a variant thereof, that contains all of the elements normally contained in one or more naturally occurring, wild type SLC15A2 polynucleotide or polypeptide sequences. For example, a full length SLC15A2 nucleic acid will typically comprise all of the exons that encode for the full length, naturally occurring protein. The “full length” may be prior to, or after, various stages of post-translation processing or splicing, including alternative splicing.

“Biological sample” as used herein is a sample of biological tissue or fluid that contains nucleic acids or polypeptides, e.g., of an SLC15A2 protein, polynucleotide or transcript. Such samples include, but are not limited to, tissue isolated from primates, e.g., humans, or rodents, e.g., mice, and rats. Biological samples may also include sections of tissues such as biopsy and autopsy samples, frozen sections taken for histologic purposes, blood, plasma, serum, sputum, stool, tears, mucus, hair, skin, etc. Biological samples also include explants and primary and/or transformed cell cultures derived from patient tissues. A biological sample is typically obtained from a eukaryotic organism, most preferably a mammal such as a primate, e.g., chimpanzee or human; cow; dog; cat; a rodent, e.g., guinea pig, rat, mouse; rabbit; or a bird; reptile; or fish.

“Providing a biological sample” means to obtain a biological sample for use in methods described in this invention. Most often, this will be done by removing a sample of cells from an animal, but can also be accomplished by using previously isolated cells (e.g., isolated by another person, at another time, and/or for another purpose), or by performing the methods of the invention in vivo. Archival tissues, having treatment or outcome history, will be particularly useful.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., Altschul et al., Nuc. Acids Res. 25:3389-3402 (1977) and Altschul et al., J. Mol. Biol. 215:403-410 (1990)). Such sequences are then said to be “substantially identical.” This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. Log values may be large negative numbers, e.g., 5, 10, 20, 30, 40, 40, 70, 90, 110, 150, 170, etc.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

A “host cell” is a naturally occurring cell or a transformed cell that contains an expression vector and supports the replication or expression of the expression vector. Host cells may be cultured cells, explants, cells in vivo, and the like. Host cells may be prokaryotic cells such as E. coli, or eukaryotic cells such as yeast, insect, amphibian, or mammalian cells such as CHO, HeLa, and the like (see, e.g., the American Type Culture Collection catalog or web site).

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG, and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor & Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that often form a compact unit of the polypeptide and are typically 25 to approximately 500 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed, usually by the noncovalent association of independent tertiary units. Anisotropic terms are also known as energy terms.

A “label” or a “detectable moiety” is a composition detectable by spectroscopic, photochemical, biochemical, immunochemical, chemical, or other physical means. For example, useful labels include fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The radioisotope may be, for example, ³H, ¹⁴C, ³²P, ³⁵S, or ¹²⁵I. In some cases, particularly using antibodies against the proteins of the invention, the radioisotopes are used as toxic moieties, as described below. The labels may be incorporated into the SLC15A2 nucleic acids, proteins and antibodies at any position. A method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982). The lifetime of radiolabeled peptides or radiolabeled antibody compositions may extended by the addition of substances that stabilize the radiolabeled peptide or antibody and protect it from degradation. A substance or combination of substances that stabilize the radiolabeled peptide or antibody may be used including those substances disclosed in U.S. Pat. No. 5,961,955.

An “effector,” also referred to herein as an “effector moiety” or an “effector component” is a molecule that is bound (or linked, or conjugated), either covalently, through a linker or a chemical bond, or noncovalently, through ionic, van der Waals, electrostatic, or hydrogen bonds, to an antibody. An “effector” may be a variety of molecules including, e.g., detection moieties including radioactive compounds, fluorescent compounds, an enzyme or substrate, tags such as epitope tags, a toxin; activatable moieties, a chemotherapeutic agent; a chemoattractant, a lipase; an antibiotic; or a radioisotope emitting “hard”, e.g., beta radiation.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, e.g., through the expression of a recombinant nucleic acid as depicted above.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “specifically (or selectively) binds” to an antibody or “specifically (or selectively) immunoreactive with,” when referring to a protein or peptide, refers to a binding reaction that is determinative of the presence of the protein, in a heterogeneous population of proteins and other biologics. Thus, under designated immunoassay conditions, the specified antibodies bind to a particular protein sequences at least two times the background and more typically more than 10 to 100 times background.

Specific binding to an antibody under such conditions requires an antibody that is selected for its specificity for a particular protein. For example, polyclonal antibodies raised to a particular protein, polymorphic variants, alleles, orthologs, and conservatively modified variants, or splice variants, or portions thereof, can be selected to obtain only those polyclonal antibodies that are specifically immunoreactive with SLC15A2 and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

“Tumor cell” refers to precancerous, cancerous, and normal cells in a tumor.

“Cancer cells,” “transformed” cells or “transformation” in tissue culture, refers to spontaneous or induced phenotypic changes that do not necessarily involve the uptake of new genetic material. Although transformation can arise from infection with a transforming virus and incorporation of new genomic DNA, or uptake of exogenous DNA, it can also arise spontaneously or following exposure to a carcinogen, thereby mutating an endogenous gene. Transformation is associated with phenotypic changes, such as immortalization of cells, aberrant growth control, nonmorphological changes, and/or malignancy (see, Freshney, Culture of Animal Cells a Manual of Basic Technique (3rd ed. 1994)).

### Expression of SLC15A2 Polypeptides from Nucleic Acids

Nucleic acids of the invention can be used to make a variety of expression vectors to express SLC15A2 polypeptides which can then be used to raise antibodies of the invention, as described below. Expression vectors and recombinant DNA technology are well known to those of skill in the art and are used to express proteins. The expression vectors may be either self-replicating extrachromosomal vectors or vectors which integrate into a host genome. Generally, these expression vectors include transcriptional and translational regulatory nucleic acid operably linked to the nucleic acid encoding the SLC15A2 protein. The term “control sequences” refers to DNA sequences used for the expression of an operably linked coding sequence in a particular host organism. Control sequences that are suitable for prokaryotes, e.g., include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is typically accomplished by ligation at convenient restriction sites. If such sites do not exist, synthetic oligonucleotide adaptors or linkers are used in accordance with conventional practice. Transcriptional and translational regulatory nucleic acid will generally be appropriate to the host cell used to express the SLC15A2 protein. Numerous types of appropriate expression vectors, and suitable regulatory sequences are known in the art for a variety of host cells.

In general, transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, an expression vector may comprise additional elements. For example, the expression vector may have two replication systems, thus allowing it to be maintained in two organisms, e.g. in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art (e.g., Fernandez & Hoeffler, supra).

In addition, in a preferred embodiment, the expression vector contains a selectable marker gene to allow the selection of transformed host cells. Selection genes are well known in the art and will vary with the host cell used.

The SLC15A2 proteins of the present invention are produced by culturing a host cell transformed with an expression vector containing nucleic acid encoding an SLC15A2 protein, under the appropriate conditions to induce or cause expression of the SLC15A2 protein. Conditions appropriate for SLC15A2 protein expression will vary with the choice of the expression vector and the host cell, and will be easily ascertained by one skilled in the art through routine experimentation or optimization. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect and animal cells, including mammalian cells. Of particular interest are Saccharomyces cerevisiae and other yeasts, E. coli, Bacillus subtilis, Sf9 cells, C129 cells, 293 cells, Neurospora, BHK, CHO, COS, HeLa cells, HUVEC (human umbilical vein endothelial cells), THP1 cells (a macrophage cell line) and various other human cells and cell lines.

In a preferred embodiment, the SLC15A2 proteins are expressed in mammalian cells. Mammalian expression systems are also known in the art, and include retroviral and adenoviral systems. One expression vector system is a retroviral vector system such as is generally described in PCT/US97/01019 and PCT/US97/01048, both of which are hereby expressly incorporated by reference. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter (see, e.g., Fernandez & Hoeffler, supra). Typically, transcription termination and polyadenylation sequences recognized by mammalian cells are regulatory regions located 3′ to the translation stop codon and thus, together with the promoter elements, flank the coding sequence. Examples of transcription terminator and polyadenylation signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In some embodiments, SLC15A2 proteins are expressed in bacterial systems. Bacterial expression systems are well known in the art. Promoters from bacteriophage may also be used and are known in the art. In addition, synthetic promoters and hybrid promoters are also useful; e.g., the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the SLC15A2 protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others. The bacterial expression vectors are transformed into bacterial host cells using techniques well known in the art, such as calcium chloride treatment, electroporation, and others.

In one embodiment, SLC15A2 polypeptides are produced in insect cells. Expression vectors for the transformation of insect cells, and in particular, baculovirus-based expression vectors, are well known in the art.

SLC15A2 polypeptides can also be produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

The SLC15A2 polypeptides may also be made as a fusion protein, using techniques well known in the art. Thus, e.g., for the creation of monoclonal antibodies, if the desired epitope is small, the SLC15A2 protein may be fused to a carrier protein to form an immunogen. Alternatively, the SLC15A2 protein may be made as a fusion protein to increase expression, or for other reasons. For example, when the SLC15A2 protein is an SLC15A2 peptide, the nucleic acid encoding the peptide may be linked to other nucleic acid for expression purposes.

The SLC15A2 polypeptides are typically purified or isolated after expression. SLC15A2 proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the SLC15A2 protein may be purified using a standard anti-SLC15A2 protein antibody column. Ultrafiltration and diafiltration techniques, in conjunction with protein concentration, are also useful. For general guidance in suitable purification techniques, see Scopes, Protein Purification (1982). The degree of purification necessary will vary depending on the use of the SLC15A2 protein. In some instances no purification will be necessary.

One of skill will recognize that the expressed protein need not have the wild-type SLC15A2 sequence but may be derivative or variant as compared to the wild-type sequence. These variants typically fall into one or more of three classes: substitutional, insertional or deletional variants. These variants ordinarily are prepared by site specific mutagenesis of nucleotides in the DNA encoding the protein, using cassette or PCR mutagenesis or other techniques well known in the art, to produce DNA encoding the variant, and thereafter expressing the DNA in recombinant cell culture as outlined above. However, variant protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the SLC15A2 protein amino acid sequence. The variants typically exhibit the same qualitative biological activity as the naturally occurring analogue, although variants can also be selected which have modified characteristics as will be more fully outlined below.

SLC15A2 polypeptides of the present invention may also be modified in a way to form chimeric molecules comprising an SLC15A2 polypeptide fused to another, heterologous polypeptide or amino acid sequence. In one embodiment, such a chimeric molecule comprises a fusion of the SLC15A2 polypeptide with a tag polypeptide which provides an epitope to which an anti-tag antibody can selectively bind. The epitope tag is generally placed at the amino-or carboxyl-terminus of the SLC15A2 polypeptide. The presence of such epitope-tagged forms of an SLC15A2 polypeptide can be detected using an antibody against the tag polypeptide. Also, provision of the epitope tag enables the SLC15A2 polypeptide to be readily purified by affinity purification using an anti-tag antibody or another type of affinity matrix that binds to the epitope tag. In an alternative embodiment, the chimeric molecule may comprise a fusion of an SLC15A2 polypeptide with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; HIS6 and metal chelation tags, the flu HA tag polypeptide and its antibody 12CA5 (Field et al., Mol. Cell. Biol. 8:2159-2165 (1988)); the c-myc tag and the 8F9, 3C7, 6E10, G4, B7 and 9E10 antibodies thereto (Evan et al., Molecular and Cellular Biology 5:3610-3616 (1985)); and the Herpes Simplex virus glycoprotein D (gD) tag and its antibody (Paborsky et al., Protein Engineering 3(6):547-553 (1990)). Other tag polypeptides include the FLAG-peptide (Hopp et al., BioTechnology 6:1204-1210 (1988)); the KT3 epitope peptide (Martin et al., Science 255:192-194 (1992)); tubulin epitope peptide (Skinner et al., J. Biol. Chem. 266:15163-15166 (1991)); and the T7 gene 10 protein peptide tag (Lutz-Freyermuth et al., Proc. Natl. Acad. Sci. USA 87:6393-6397 (1990)).

### Antibodies to Cancer or Fibrosis Proteins

Once the SLC15A2 protein is produced, it is used to generate antibodies, e.g., for immunotherapy or immunodiagnosis. In some embodiments of the invention, the antibodies recognize the same epitope as the CDRs shown in Table 2. The ability of a particular antibody to recognize the same epitope as another antibody is typically determined by the ability of one antibody to competitively inhibit binding of the second antibody to the antigen. Any of a number of competitive binding assays can be used to measure competition between two antibodies to the same antigen. An exemplary assay is a Biacore assay as described in the Examples, below. Briefly in these assays, binding sites can be mapped in structural terms by testing the ability of interactions, e.g. different antibodies, to inhibit the binding of another. Injecting two consecutive antibody samples in sufficient concentration can identify pairs of competing antibodies for the same binding epitope. The antibody samples should have the potential to reach a significant saturation with each injection. The net binding of the second antibody injection is indicative for binding epitope analysis. Two response levels can be used to describe the boundaries of perfect competition versus non-competing binding due to distinct epitopes. The relative amount of binding response of the second antibody injection relative to the binding of identical and distinct binding epitopes determines the degree of epitope overlap.

Other conventional immunoassays known in the art can be used in the present invention. For example, antibodies can be differentiated by the epitope to which they bind using a sandwich ELISA assay. This is carried out by using a capture antibody to coat the surface of a well. A subsaturating concentration of tagged-antigen is then added to the capture surface. This protein will be bound to the antibody through a specific antibody:epitope interaction. After washing a second antibody, which has been covalently linked to a detectable moiety (e.g., HRP, with the labeled antibody being defined as the detection antibody) is added to the ELISA. If this antibody recognizes the same epitope as the capture antibody it will be unable to bind to the target protein as that particular epitope will no longer be available for binding. If however this second antibody recognizes a different epitope on the target protein it will be able to bind and this binding can be detected by quantifying the level of activity (and hence antibody bound) using a relevant substrate. The background is defined by using a single antibody as both capture and detection antibody, whereas the maximal signal can be established by capturing with an antigen specific antibody and detecting with an antibody to the tag on the antigen. By using the background and maximal signals as references, antibodies can be assessed in a pair-wise manner to determine epitope specificity.

A first antibody is considered to competitively inhibit binding of a second antibody, if binding of the second antibody to the antigen is reduced by at least 30%, usually at least about 40%, 50%, 60% or 75%, and often by at least about 90%, in the presence of the first antibody using any of the assays described above.

Methods of preparing polyclonal antibodies are known to the skilled artisan (e.g., Coligan, supra; and Harlow & Lane, supra). Polyclonal antibodies can be raised in a mammal, e.g., by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the figures or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler & Milstein, Nature 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Table 1, a fragment thereof, or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, pp. 59-103 (1986)). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In some embodiments the antibodies to the SLC15A2 proteins are chimeric or humanized antibodies. As noted above, humanized forms of antibodies are chimeric immunoglobulins in which residues from a complementary determining region (CDR) of human antibody are replaced by residues from a CDR of a non-human species such as mouse, rat or rabbit having the desired specificity, affinity and capacity.

Human antibodies can be produced using various techniques known in the art, including phage display libraries (Hoogenboom & Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, p. 77 (1985) and Boerner et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

In some embodiments, the antibody is a single chain Fv (scFv). The V_{H} and the V_{L} regions of a scFv antibody comprise a single chain which is folded to create an antigen binding site similar to that found in two chain antibodies. Once folded, noncovalent interactions stabilize the single chain antibody. While the V_{H} and V_{L} regions of some antibody embodiments can be directly joined together, one of skill will appreciate that the regions may be separated by a peptide linker consisting of one or more amino acids. Peptide linkers and their use are well-known in the art. See, e.g., Huston et al., Proc. Nat'l Acad. Sci. USA 8:5879 (1988); Bird et al., Science 242:4236 (1988); Glockshuber et al., Biochemistry 29:1362 (1990); U.S. Pat. No. 4,946,778, U.S. Pat. No. 5,132,405 and Stemmer et al., Biotechniques 14:256-265 (1993). Generally the peptide linker will have no specific biological activity other than to join the regions or to preserve some minimum distance or other spatial relationship between the V_{H} and V_{L}. However, the constituent amino acids of the peptide linker may be selected to influence some property of the molecule such as the folding, net charge, or hydrophobicity. Single chain Fv (scFv) antibodies optionally include a peptide linker of no more than 50 amino acids, generally no more than 40 amino acids, preferably no more than 30 amino acids, and more preferably no more than 20 amino acids in length. In some embodiments, the peptide linker is a concatamer of the sequence Gly-Gly-Gly-Gly-Ser, preferably 2, 3, 4, 5, or 6 such sequences. However, it is to be appreciated that some amino acid substitutions within the linker can be made. For example, a valine can be substituted for a glycine.

Methods of making scFv antibodies have been described. See, Huse et al., supra; Ward et al. supra; and Vaughan et al., supra. In brief, mRNA from B-cells from an immunized animal is isolated and cDNA is prepared. The cDNA is amplified using primers specific for the variable regions of heavy and light chains of immunoglobulins. The PCR products are purified and the nucleic acid sequences are joined. If a linker peptide is desired, nucleic acid sequences that encode the peptide are inserted between the heavy and light chain nucleic acid sequences. The nucleic acid which encodes the scFv is inserted into a vector and expressed in the appropriate host cell. The scFv that specifically bind to the desired antigen are typically found by panning of a phage display library. Panning can be performed by any of several methods. Panning can conveniently be performed using cells expressing the desired antigen on their surface or using a solid surface coated with the desired antigen. Conveniently, the surface can be a magnetic bead. The unbound phage are washed off the solid surface and the bound phage are eluted.

Finding the antibody with the highest affinity is dictated by the efficiency of the selection process and depends on the number of clones that can be screened and the stringency with which it is done. Typically, higher stringency corresponds to more selective panning. If the conditions are too stringent, however, the phage will not bind. After one round of panning, the phage that bind to SLC15A2 coated plates or to cells expressing SLC15A2 on their surface are expanded in E. Coli and subjected to another round of panning. In this way, an enrichment of many fold occurs in 3 rounds of panning. Thus, even when enrichment in each round is low, multiple rounds of panning will lead to the isolation of rare phage and the genetic material contained within which encodes the scFv with the highest affinity or one which is better expressed on phage.

Regardless of the method of panning chosen, the physical link between genotype and phenotype provided by phage display makes it possible to test every member of a cDNA library for binding to antigen, even with large libraries of clones.

In one embodiment, the antibodies are bispecific antibodies. Bispecific antibodies are monoclonal, preferably human or humanized, antibodies that have binding specificities for at least two different antigens or that have binding specificities for two epitopes on the same antigen. In one embodiment, one of the binding specificities is for the SLC15A2 protein, the other one is for another cancer antigen. Alternatively, tetramer-type technology may create multivalent reagents.

In some embodiments, the antibodies to SLC15A2 protein are capable of reducing or eliminating cells expressing SLC15A2 (e.g., ovarian cancer cells, cervical cancer cells, prostate cancer cells, uterine cancer cells, lung cancer cells, lung fibrosis cells, and glioblastoma cells). Generally, at least a 25% decrease in activity, growth, size or the like is preferred, with at least about 50% being particularly preferred and about a 95-100% decrease being especially preferred.

By immunotherapy is meant treatment of cancer with an antibody raised against SLC15A2 proteins. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen (e.g., SLC15A2 or DNA encoding it) to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen, leading to an immune response.

In some embodiments, the antibody is conjugated to an effector moiety (i.e. an effector component). The effector moiety can be any number of molecules, including labeling moieties such as radioactive labels or fluorescent labels, or can be a therapeutic moiety. In one aspect the therapeutic moiety is a small molecule that modulates the activity of the SLC15A2 protein. In another aspect the therapeutic moiety modulates the activity of molecules associated with or in close proximity to the SLC15A2 protein.

In other embodiments, the therapeutic moiety is a cytotoxic agent. In this method, targeting the cytotoxic agent to cancer tissue or cells, results in a reduction in the number of afflicted cells, thereby reducing symptoms associated with the cancer. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin, auristatin and the like. Cytotoxic agents also include radiochemicals made by conjugating radioisotopes to antibodies raised against ovarian cancer proteins, or binding of a radionuclide to a chelating agent that has been covalently attached to the antibody. Targeting the therapeutic moiety to transmembrane cancer proteins not only serves to increase the local concentration of therapeutic moiety in the afflicted area, but also serves to reduce deleterious side effects that may be associated with the therapeutic moiety.

### Binding Affinity of Antibodies of the Invention

Binding affinity for a target antigen is typically measured or determined by standard antibody-antigen assays, such as Biacore competitive assays, saturation assays, or immunoassays such as ELISA or RIA.

Such assays can be used to determine the dissociation constant of the antibody. The phrase “dissociation constant” refers to the affinity of an antibody for an antigen. Specificity of binding between an antibody and an antigen exists if the dissociation constant (K_{D}=1/K, where K is the affinity constant) of the antibody is <1 μM, preferably <100 nM, and most preferably <0.1 nM. Antibody molecules will typically have a K_{D} in the lower ranges. K_{D}=[Ab-Ag]/[Ab][Ag] where [Ab] is the concentration at equilibrium of the antibody, [Ag] is the concentration at equilibrium of the antigen and [Ab-Ag] is the concentration at equilibrium of the antibody-antigen complex. Typically, the binding interactions between antigen and antibody include reversible noncovalent associations such as electrostatic attraction, Van der Waals forces and hydrogen bonds.

The antibodies of the invention specifically bind to SLC15A2 proteins. By “specifically bind” herein is meant that the antibodies bind to the protein with a K_{D} of at least about 0.1 mM, more usually at least about 1 μM, preferably at least about 0.1 μM or better, and most preferably, 0.01 μM or better.

Selectivity of an antibody refers to how selective it is in distinguishing between related proteins.

### Immunoassays

The antibodies of the invention can be used to detect SLC15A2 or SLC15A2 expressing cells using any of a number of well recognized immunological binding assays (see, e.g., U.S. Pat. Nos. 4,366,241; 4,376,110; 4,517,288; and 4,837,168). For a review of the general immunoassays, see also Methods in Cell Biology, Vol. 37, Asai, ed. Academic Press, Inc. New York (1993); Basic and Clinical Immunology 7th Edition, Stites & Terr, eds. (1991).

Thus, the present invention provides methods of detecting cells that express SLC15A2. In one method, a biopsy is performed on the subject and the collected tissue is tested in vitro. The tissue or cells from the tissue is then contacted, with an anti-SLC15A2 antibody of the invention. Any immune complexes which result indicate the presence of an SLC15A2 protein in the biopsied sample. To facilitate such detection, the antibody can be radiolabeled or coupled to an effector component which is a detectable label, such as a radiolabel. In another method, the cells can be detected in vivo using typical imaging systems. Then, the localization of the label is determined by any of the known methods for detecting the label. A conventional method for visualizing diagnostic imaging can be used. For example, paramagnetic isotopes can be used for MRI. Internalization of the antibody may be important to extend the life within the organism beyond that provided by extracellular binding, which will be susceptible to clearance by the extracellular enzymatic environment coupled with circulatory clearance.

SLC15A2 proteins can also be detected using standard immunoassay methods and the antibodies of the invention. Standard methods include, for example, radioimmunoassay, sandwich immunoassays (including ELISA), immunofluorescence assays, Western blot, affinity chromatography (affinity ligand bound to a solid phase), and in situ detection with labeled antibodies.

### Administration of Pharmaceutical and Vaccine Compositions

The antibodies of the invention can be formulated in pharmaceutical compositions. Thus, the invention also provide methods and compositions for administering a therapeutically effective dose of an anti-SLC15A2 antibody. The exact dose will depend on the purpose of the treatment, and will be ascertainable by one skilled in the art using known techniques (e.g., Ansel et al., Pharmaceutical Dosage Forms and Drug Delivery; Lieberman, Pharmaceutical Dosage Forms (vols. 1-3, 1992), Dekker, ISBN 0824770846, 082476918X, 0824712692, 0824716981; Lloyd, The Art, Science and Technology of Pharmaceutical Compounding (1999); and Pickar, Dosage Calculations (1999)). As is known in the art, adjustments for ovarian cancer degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art. U.S. patent application Ser. No. 09/687,576, further discloses the use of compositions and methods of diagnosis and treatment in ovarian cancer is hereby expressly incorporated by reference.

A “patient” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

The administration of the antibodies of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly.

The pharmaceutical compositions of the present invention comprise an antibody of the invention in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that antibodies when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The compositions for administration will commonly comprise an antibody of the invention dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art, e.g., Remington's Pharmaceutical Science and Goodman and Gillman, The Pharmacological Basis of Therapeutics, supra.

The compositions containing antibodies of the invention can be administered for therapeutic or prophylactic treatments. In therapeutic applications, compositions are administered to a patient suffering from a disease (e.g., a cancer) in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the agents of this invention to effectively treat the patient. An amount of modulator that is capable of preventing or slowing the development of cancer in a mammal is referred to as a “prophylactically effective dose.” The particular dose required for a prophylactic treatment will depend upon the medical condition and history of the mammal, the particular cancer being prevented, as well as other factors such as age, weight, gender, administration route, efficiency, etc. Such prophylactic treatments may be used, e.g., in a mammal who has previously had cancer to prevent a recurrence of the cancer, or in a mammal who is suspected of having a significant likelihood of developing cancer.

It will be appreciated that the present ovarian cancer protein-modulating compounds can be administered alone or in combination with additional ovarian cancer modulating compounds or with other therapeutic agent, e.g., other anti-cancer agents or treatments.

### Kits for Use in Diagnostic and/or Prognostic Applications

For use in diagnostic, research, and therapeutic applications suggested above, kits are also provided by the invention. In the diagnostic and research applications such kits may include any or all of the following: assay reagents, buffers, and SLC15A2-specific antibodies of the invention. A therapeutic product may include sterile saline or another pharmaceutically acceptable emulsion and suspension base.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

### Example 1

### Production of Selective Anti-SLC15A2 Monoclonal Antibodies

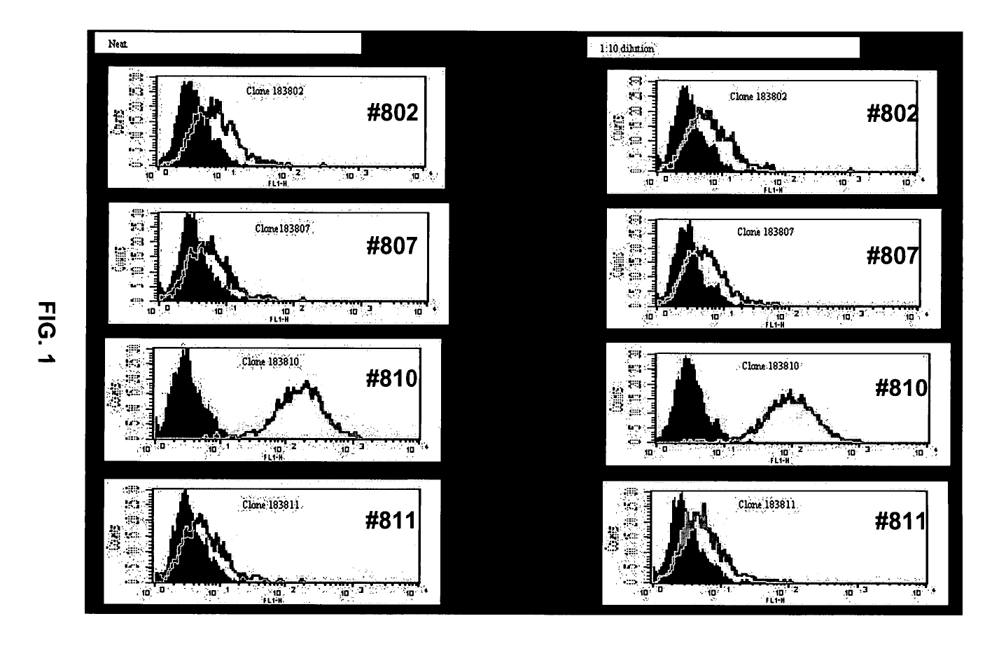

3T12 cells and Calu6 cancer cells were transfected with a CHEF expression vector containing the cDNA encoding the SLCA15A2 protein. Stable SLC15A2 expressing cells were generated by G418 selection. The 3T12 cells expressing SLC15A2 were then used to immunize mice to generate anti-SLC15A2 monoclonal antibodies. After several rounds of immunization, spleens were harvested to generate antibody producing hybridomas. Hybridomas that produce antibodies that specifically bind to the extracellular region of SLC15A2 were then identified using fluorescence activated cell sorting (FACS) (see FIG. 1). Three hybridoma clones (clones 810, 811 and 824) were selected for further study. As shown in FIG. 1, Clone 810 supernatant showed the strongest FACS profile among the three, suggesting that this clone produced the antibody with the highest affinity.

The nucleotide sequences of the heavy and light chain variable regions for the antibodies PDO5#810 and PDO5#811 produced by clones 810 and 811, respectively, were determined using standard techniques. The V_{H} and V_{L} region nucleotide and derived amino acid sequences are listed in Table 2 as SEQ ID NOs: 3-10. The corresponding CDR region sequences of each antibody are depicted as underlined and bolded in Table 2.

### Example 2

### Specific Killing of SLC15A2-Expressing Cells Using Anti-SLC15A2 Specific Antibodies as a Toxin Targeting Agent

This study was designed to determine the value of the H+/peptide transporter SLC15A2 as a therapeutic target for prostate cancer treatment. To test antibody-mediated killing of cancer cells, parental Calu6 cells, which do not express SLC15A2, and SLC15A2 expressing Calu6 cells were plated in 96 wells and were allowed to adhere overnight. The next day anti-SLC15A2 (clones 810, 811 and 824) antibodies or isotype control antibodies, all in the form of hybridoma tissue culture supernatant, were added to the cells. Cells were then incubated with secondary anti-mouse Ig antibodies conjugated to the ribosome toxin saporin. After four days in culture, cell growth and cell killing were assessed using an MTT assay. Saporin is a biological entity that can kill cells only when actively transported into the cells.

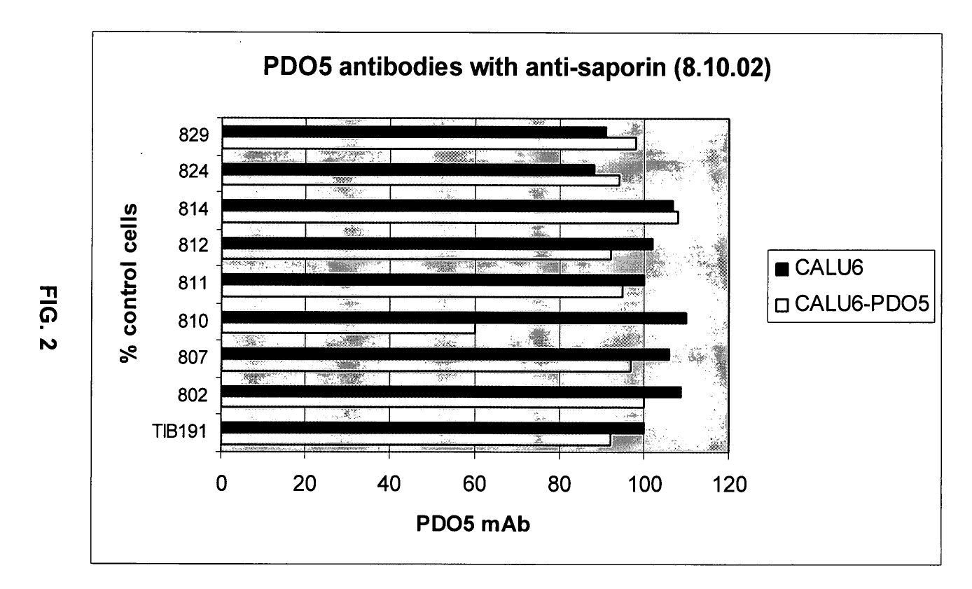

The results show that antibody PDO5#810, which specifically targets SLC5A2, binds to SLC15A2 on the cell surface and internalizes together with the anti-mouse Ig-saporin conjugate (see FIG. 2). This results in effective cell killing of SLC15A2 expressing cells, but not in the death of parental Calu6 cells. Combined with the prostate cancer-specific expression of SLC15A2, this data confirms that SLC15A2 is a potential therapeutic target for the treatment of prostate cancer.

### Example 3

### Effect of Antibodies on Tumor Cell Growth In Vivo

Animal studies are conducted using SCID mice immunized with an appropriate human tumor cell line or transfected cell line, e.g., CALU6. A cell line is selected that expresses the antigen recognized by SLC15A2 antibodies, the protein and nucleic acid sequences of which are provided in Table 1 as SEQ ID NOs 1 and 2.

To initiate tumor growth in vivo SCID mice are injected with the cell line and tumors are allowed to grow. When tumors reach a size of between 50-100 mm³, animals are distributed into groups and subjected to treatment with either a.) an isotype control antibody, b.) one or more SLC15A2 antibodies, or c.) SLC15A2 antibodies in conjunction with the chemotherapeutic agents, e.g., paclitaxel and carboplatin.

Antibodies are administered, e.g., every 2 days at a dose of 10 mg/kg. For the antibody plus chemotherapy group, chemotherapies may be administered together at 4 day intervals for 4 doses and the antibodies are administered at 10 mg/kg at 4 day intervals for 3 doses. Tumor size is measured, e.g., twice weekly for 20 days.

The tumor volumes are compared among mice receiving treatment with the isotype control antibody and the mice receiving treatment with SLC15A2 antibody, with a significant reduction in tumor volume resulting from the good therapeutic agents.

Furthermore, since the effects of SLC15A2 antibodies and chemotherapeutic agents on tumor volume reduction are additive, the therapeutic use of SLC15A2 antibodies will reduce the amounts of chemotherapeutics needed for effective reduction of tumor size in cancer patients and this in turn, will reduce patient suffering due to toxic side effects of chemotherapeutic agents.

### Example 4

### SLC15A2 Expression in Prostate Cancer

In an effort to identify potential therapeutic targets in prostate cancer, gene expression of 74 prostate cancers was compared to 347 normal adult tissues representing 58 different organs. The goal was to look for genes that are up-regulated in prostate cancer and are localized to the cell surface for antibody accessibility, but have little to no expression in vital organs to minimize undesirable side effects of a targeted antibody. Genes with the desired expression profile were triaged by extensive bioinformatic analysis to determine their structural and functional classification, and determine their potential for cell surface localization.

### DNA Microarray Analysis

Tumor tissue from 74 patients treated with radical prostatectomy for clinically localized prostate cancer and more than 300 non-malignant adult tissues and organs were collected were collected and processed for gene expression profiling using the Eos Hu03, an Affymetrix GeneChip as previously published (Henshall et al., Cancer Res. 63:4196-4203 (2003); Henshall et al., Oncogene 22:6005-6012 (2003); Bhaskar et al., Cancer Res. 63:6387-6394 (2003)). The clinical parameters of the patient cohort were previously described in detail (Henshall et al., Cancer Res. 63:4196-4203 (2003); Henshall et al., Oncogene 22:6005-6012 (2003); Bhaskar et al., Cancer Res. 63:6387-6394 (2003)). Gene array data on the prostate cancer cohort, data mining methods for prostate cancer antigens and bioinformatics analysis were also previously described (Henshall et al., Cancer Res. 63:4196-4203 (2003); Henshall et al., Oncogene 22:6005-6012 (2003); Bhaskar et al., Cancer Res. 63:6387-6394 (2003)).

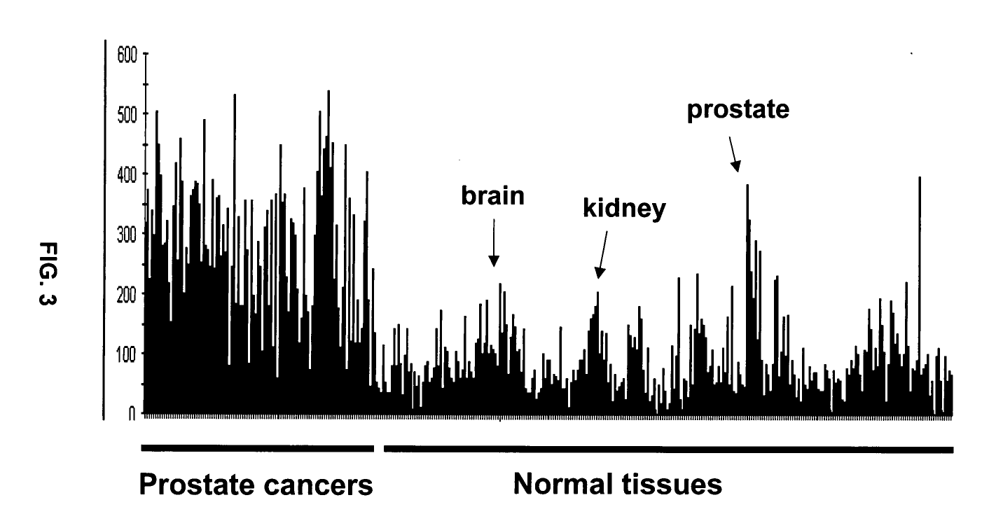

The SLC15A2 gene (NCBI reference sequence no. NM_{—}021082.2; Ref. Liu et al, Biochim. Biophys. Acta 1235:461-466 (1995)) displayed all the desired characteristics. As shown in FIG. 3, the SLC15A2 mRNA expression level in prostate cancer significantly exceeds expression in normal body tissues. Expression was also detected in brain, kidney and normal prostate. Among non-prostate cancer tissues, higher than normal expression of SLC15A2 was detected in lung, uterine, ovarian and cervical cancers, as well as in glioblastoma (data not shown). The gene chip expression data was also confirmed by TaqMan® analysis of the same samples (data not shown). Bioinformatics analysis of the SLC15A2 gene sequence suggested that the protein product contains multiple transmembrane domains and is predicted to locate to the plasma membrane, making it a suitable candidate target for therapeutic antibodies.

IHC Analysis

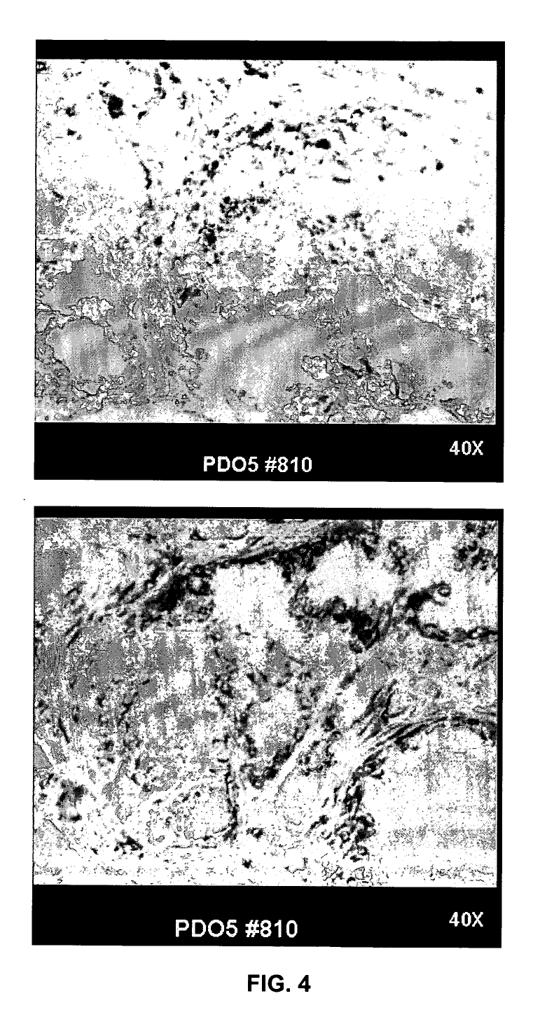

To confirm protein expression of SLC15A2 in human tissues, fresh frozen sections of human prostate from prostate cancer patients were stained with monoclonal antibody PDO5 #810. The results show PDO5 #810 clearly recognize SLC15A2 protein in the prostate secretory epithelium of 2 separate prostate cancer patients (FIG. 4). This indicates that SLC15A2 protein expression parallels the gene expression profiles detected by DNA microarray analysis.

TABLE 1
DNA AND PROTEIN SEQUENCES OF SLC15A2

SEQ ID NO: 1 SLC15A2 DNA SEQUENCE
gaggagagag agagagtaag gagccagccA TGAATCCTTT CCAGAAAAAT GAGTCCAAGG
AAACTCTTTT TTCACCTGTC TCCATTGAAG AGGTACCACC TCGACCACCT AGCCCTCCAA
AGAAGCCATC TCCGACAATC TGTGGCTCCA ACTATCCACT GAGCATTGCC TTCATTGTGG
TGAATGAATT CTGCGAGCGC TTTTCCTATT ATGGAATGAA AGCTGTGCTG ATCCTGTATT
TCCTGTATTT CCTGCACTGG AATGAAGATA CCTCCACATC TATATACCAT GCCTTCAGCA
GCCTCTGTTA TTTTACTCCC ATCCTGGGAG CAGCCATTGC TGACTCGTGG TTGGGAAAAT
TCAAGACAAT CATCTATCTC TCCTTGGTGT ATGTGCTTGG CCATGTGATC AAGTCCTTGG
GTGCCTTACC AATACTGGGA GGACAAGTCG TACACACAGT CCTATCATTG ATCGGCCTGA
GTCTAATAGC TTTGGGGACA GGAGGCATCA AACCCTGTGT GGCAGCTTTT GGTGGAGACC
AGTTTGAAGA AAAACATGCA GAGGAACGGA CTAGATACTT CTCAGTCTTC TACCTGTCCA
TCAATGCAGG GAGCTTGATT TCTACATTTA TCACACCCAT GCTGAGAGGA GATGTGCAAT
GTTTTGGAGA AGACTGCTAT GCATTGGCTT TTGGAGTTCC AGGACTGCTC ATGGTAATTG
CACTTGTTGT GTTTGCAATG GGAAGCAAAA TATACAATAA ACCACCCCCT GAAGGAAACA
TAGTGGCTCA AGTTTTCAAA TGTATCTGGT TTGCTATTTC CAATCGTTTC AAGAACCGTT
CTGGAGACAT TCCAAAGCGA CAGCACTGGC TAGACTGGGC AGCTGAGAAA TATCCAAAGC
AGCTCATTAT GGATGTAAAG GCACTGACCA GGGTACTATT CCTTTATATC CCATTGCCCA
TGTTCTGGGC TCTTTTGGAT CAGCAGGGTT CACGATGGAC TTTGCAAGCC ATCAGGATGA
ATAGGAATTT GGGGTTTTTT GTGCTTCAGC CGGACCAGAT GCAGGTTCTA AATCCCTTTC
TGGTTCTTAT CTTCATCCCG TTGTTTGACT TTGTCATTTA TCGTCTGGTC TCCAAGTGTG
GAATTAACTT CTCATCACTT AGGAAAATGG CTGTTGGTAT GATCCTAGCG TGCCTGGCAT
TTGCAGTTGC GGCAGCTGTA GAGATAAAAA TAAATGAAAT GGCCCCAGCC CAGTCAGGTC
CCCAGGAGGT TTTCCTACAA GTCTTGAATC TGGCAGATGA TGAGGTGAAG GTGACAGTGG
TGGGAAATGA AAACAATTCT CTGTTGATAG AGTCCATCAA ATCCTTTCAG AAAACACCAC
ACTATTCCAA ACTGCACCTG AAAACAAAAA GCCAGGATTT TCACTTCCAC CTGAAATATC
ACAATTTGTC TCTCTACACT GAGCATTCTG TGCAGGAGAA GAACTGGTAC AGTCTTGTCA
TTCGTGAAGA TGGGAACAGT ATCTCCAGCA TGATGGTAAA GGATACAGAA AGCAAAACAA
CCAATGGGAT GACAACCGTG AGGTTTGTTA ACACTTTGCA TAAAGATGTC AACATCTCCC
TGAGTACAGA TACCTCTCTC AATGTTGGTG AAGACTATGG TGTGTCTGCT TATAGAACTG
TGCAAAGAGG AGAATACCCT GCAGTGCACT GTAGAACAGA AGATAAGAAC TTTTCTCTGA
ATTTGGGTCT TCTAGACTTT GGTGCAGCAT ATCTGTTTGT TATTACTAAT AACACCAATC
AGGGTCTTCA GGCCTGGAAG ATTGAAGACA TTCCAGCCAA CAAAATGTCC ATTGCGTGGC
AGCTACCACA ATATGCCCTG GTTACAGCTG GGGAGGTCAT GTTCTCTGTC ACAGGTCTTG
AGTTTTCTTA TTCTCAGGCT CCCTCTAGCA TGAAATCTGT GCTCCAGGCA GCTTGGCTAT
TGACAATTGC AGTTGGGAAT ATCATCGTGC TTGTTGTGGC ACAGTTCAGT GGCCTGGTAC
AGTGGGCCGA ATTCATTTTG TTTTCCTGCC TCCTGCTGGT GATCTGCCTG ATCTTCTCCA
TCATGGGCTA CTACTATGTT CCTGTAAAGA CAGAGGATAT GCGGGGTCCA GCAGATAAGC
ACATTCCTCA CATCCAGGGG AACATGATCA AACTAGAGAC CAAGAAGACA AAACTCTGAt
gacttcctag attctgtcct gaccccaatt cctggccctg tcttgaagca ttttttttct
tctactggat tagacaagag agatagcagc atatcagagc tgatctcctc cacctttctc
caatgacaga agttccagga ctggttttcc agtacatctt taaacaaggc cccagagact
ctatgtctgc ccgtccatca gtgaactcat taaaacttgt gcagtgttgc tggagctggc
ctggtgtctc caaatgacca tgaaaataca cacgtataat ggagatcatt ctctgtgggt
atgcaaagtt atgggaattc ctttataggt aactgccatt taggactgat ggccctaatt
tttgaggtgc tgatttagag gcaaaattgc agaataacaa agaaatggta tttcaagttt
ttttttttat aagcaatgta attatgctat tcacaggggc c
SEQ ID NO: 2 SLC15A2 PROTEIN SEOUENCE
MNPFQKNESKETLFSPVSIEEVPPRPPSPPKKPSPTICGSNYPLSIAFIVVNEFCERFSYYGMKAVL
ILYFLYFLHWNEDTSTSIYHAFSSLCYFTPILGAAIADSWLGKFKTIIYLSLVYVLGHVIKSLGALP
ILGGQVVHTVLSLIGLSLIALGTGGIKPCVAAFGGDQFEEKHAEERTRYFSVFYLSINAGSLISTFI
TPMLRGDVQCFGEDCYALAFGVPGLLMVIALVVFAMGSKIYNKPPPEGNIVAQVFKCIWFAISNRFK
NRSGDIPKRQHWLDWAAEKYPKQLIMDVKALTRVLFLYIPLPMFWALLDQQGSRWTLQAIRMNRNLG
FFVLQPDQMQVLNPFLVLIFIPLFDFVIYRLVSKCGINFSSLRKMAVGMILACLAFAVAAAVEIKIN
EMAPAQSGPQEVFLQVLNLADDEVKVTVVGNENNSLLIESIKSFQKTPHYSKLHLKTKSQDFHFHLK
YHNLSLYTEHSVQEKNWYSLVIREDGNSISSMMVKDTESKTTNGMTTVRFVNTLHKDVNISLSTDTS
LNVGEDYGVSAYRTVQRGEYPAVHCRTEDKNFSLNLGLLDFGAAYLFVITNNTNQGLQAWKIEDIPA
NKMSIAWQLPQYALVTAGEVMFSVTGLEFSYSQAPSSMKSVLQAAWLLTIAVGNIIVLVVAQFSGLV
QWAEFILFSCLLLVICLIFSIMGYYYVPVKTEDMRGPADKHIPHIQGNMIKLETKKTKL

TABLE 2

Nucleotide and Protein Sequences of SLC15A2
Antibody Clones. In the Table, CDR protein
regions are shown bolded and underlined.

```
Nucleotide Sequences
SEQ ID NO: 3:
PDO5 #810 Heavy Chain Variable Region:
GAGGTCCAGCTGCAACAGTCTGGACCTGAGCTGGTGAAGCCTGGAGCTTC
AATGAAGATATCCTGCAAGGCTTCTGGTTACTCATTCACTGGCTACACCA
TGAACTGGGTGAAGCAGAGCCATGGAAAGAACCTTGAGTGGATTGGACTT
ATTAATCCTTACAATGGTGGTATTAACTACAACCAGAAGTTCAAGGGCAA
GGCCACATTAACTGTAGACAAGTCATCCAGTACAGCCTACATGGAGCTCC
TCAGTCTGACATCTGAGGACTCTGCAGTCTATTACTGTACAAGACGGGCC
TACTATGGTAACTACGGTACTATGGACTACTGGGGTCAAGGAACCTCAGT
CACCGTCTCCTCA
SEQ ID NO: 4:
PDO5 #810 Light Chain Variable Region
GAAAATGTTCTCACCCAGTCTCCAGCAAGCATGTCTGCATCTCCAGGGGA
AAAGGTCACCATGACCTGCAGTGCCAGCTCAAGTGTAAGTTACATGCACT
GGTACCAGCAGAAGTCAACCACCTCCCCCAAACTCTGGATTTATGACACA
TCCAATCTGGCTTCTGGGGTCCCAGGTCGCTTCAGTGGCAGTGGGTCTGG
AAACTCTTACTCTCTCACGATCAGCAACATGGAGGCTGAAGATGTTGCCA
CTTATTACTGTTTTCAGGGGAGTGGTTACCCACTCACGTTCGGTGCTGGG
ACCAAGCTGGAGCTGAAACGG
SEQ ID NO: 5:
PDO5 #811 Heavy Chain Variable Region
CAGGTCCAACTGCAGCAGCCTGGGGCTGAGCTGGTGAGGCCTGGGGCTTC
AGTGAAGCTGTCCTGCAAGGCTTCTGGCTACACCTTCACCAGCTACTGGT
TGAACTGGGTGAGGCAGAGGCCTGGACAAGGCCTTGAATGGATTGGTATG
ATTGATCCTTCAGACAGTGAAACTCACTACAATCAAATGTTCAAGGACAA
GGCCACATTGACTGTAGACAAGTCCTCCAGCACAGCCTACATGCAGCTCA
GCAGCCTGACATCTGAGGACTCTGCGGTCTATTACTGTACAAGTCAGGGG
GTACCGGTCCCCTTTGACTACTGGGGCCAAGGCACCACTCTCACAGTCTC
CTCA
SEQ ID NO: 6:
PDO5 #811 Light Chain Variable Region
GATGTTGTGATGACCCAAACTCCACTCTCCCTGCCTGTCAGTCTTGGAGA
TCAAGCCTCCATCTCTTGCAGATCTAGTCAGAGCCTTGTACACAGTAATG
GAAACACCTATTTACATTGGTACCTGCAGAAGCCAGGCCAGTCTCCAAAG
CTCCTGATCTACAGAGTTTCCAACCGATTTTCTGGGGTCCCAGACAGGTT
CAGTGGCAGTGGATCAGGGACAGATTTCACACTCAAGATCAGCAGAGTGG
AGGCTGAGGATCTGGGAGTTTATTTCTGCTCTCAAAGTACACATGTTCCG
TGGACGTTCGGTGGAGGCACCAAGCTGGAAATCAAACGG
PROTEIN SEQUENCES
SEQ ID NO: 7:
PDO5 #810 Heavy Chain Variable Region
EVQLQQSGPELVKPGASMKISCKASGYSFTGYTMNWVKQSHGKNLEWIGL
                                                  CDR1
INPYNGGINYNQKFKGKATLTVDKSSSTAYMELLSLTSEDSAVYYCTRRA
          CDR2
YYGNYGTMDYWGQGTSVTVSS
CDR3
SEQ ID NO: 8:
PDO5 #810 Light Chain Variable Region
ENVLTQSPASMSASPGEKVTMTCSASSSVSYMHWYQQKSTTSPKLWIYDT
                                                      CDR1
SNLASGVPGRFSGSGSGNSYSLTISNMEAEDVATYYCFQGSGYPLTFGAG
CDR2 CDR3
TKLELKR
SEQ ID NO: 9:
PDO5 #811 Heavy Chain Variable Region
QVQLQQPGAELVRPGASVKLSCKASGYTFTSYWLNWVRQRPGQGLEWIGM
                                                      CDR1
IDPSDSETHYNQMFKDKATLTVDKSSSTAYMQLSSLTSEDSAVYYCTSQG
          CDR2
VPVPFDYWGQGTTLTVSS
      CDR3
SEQ ID NO: 10:
PDO5 #811 Light Chain Variable Region
DVVMTQTPLSLPVSLGDQASISCRSSQSLVHSNGNTYLHWYLQKPGQSPK
                                                        CDR1
LLIYRVSNRFSGVPDRFSGSGSGTDFTLKISRVEAEDLGVYFCSQSTHVP
          CDR2 CDR3
WTFGGGTKLEIKR
```

It is understood that the examples described above in no way serve to limit the true scope of this invention, but rather are presented for illustrative purposes. All publications, sequences of accession numbers, and patent applications cited in this specification are herein incorporated by reference as if each individual publication or patent application were specifically and individually indicated to be incorporated by reference.

All UniGene cluster identification numbers and accession numbers herein are for the GenBank sequence database and the sequences of the accession numbers are hereby expressly incorporated by reference. GenBank is known in the art, see, e.g., Benson, D A, et al., Nucleic Acids Research 26:1-7 (1998). Sequences are also available in other databases, e.g., European Molecular Biology Laboratory (EMBL) and DNA Database of Japan (DDBJ).

What is claimed is:

1. An antibody that competitively inhibits binding of SLC15A2 polypeptide to a second antibody comprising a CDR sequence of PDO5 #810 or #811.

2. The antibody of claim 1, wherein the antibody is conjugated to an effector component.

3. The antibody of claim 2, wherein the effector component is a fluorescent label.

4. The antibody of claim 2, wherein the effector component is a radioisotope or a cytotoxic chemical.

5. The antibody of claim 4, wherein the cytotoxic chemical is auristatin.

6. The antibody of claim 1, wherein the antibody is an antibody fragment.

7. The antibody of claim 1, wherein the antibody is humanized.

8. The antibody of claim 1, wherein the antibody comprises an amino acid sequence selected from the group consisting of SEQ ID NO: 7, 8, 9 and 10.

9. The antibody of claim 1, wherein the SLC15A2 polypeptide is on a cancer or fibrosis cell.

10. A pharmaceutical composition comprising a pharmaceutically acceptable excipient and the antibody of claim 1.

11. The pharmaceutical composition of claim 10, wherein the antibody is conjugated to an effector component.

12. The pharmaceutical composition of claim 11, wherein the effector component is a fluorescent label.

13. The pharmaceutical composition of claim 11, wherein the effector component is a radioisotope or a cytotoxic chemical.

14. The pharmaceutical composition of claim 13, wherein the cytotoxic chemical is auristatin.

15. The pharmaceutical composition of claim 10, wherein the antibody is humanized.

16. The pharmaceutical composition of claim 10, wherein the antibody comprises an amino acid sequence selected from the group consisting of SEQ ID NO: 7, 8, 9 and 10.

17. A method of detecting a cancer or fibrosis cell in a biological sample from a patient, the method comprising contacting the biological sample with an antibody of claim 1.

18. The method of claim 17, wherein the cancer or fibrosis cell is selected from the group consisting of an ovarian, uterine, prostate, lung, glioblastoma, cervical, or fibrosis-associated cell.

19. The method of claim 17, wherein the antibody is conjugated to a fluorescent label.

20. A method of inhibiting proliferation of an ovarian, uterine, prostate, lung, glioblastoma, cervical, or fibrosis-associated cell, the method comprising the step of contacting the cell with an antibody of claim 1.

21. The method of claim 20, wherein the antibody is an antibody fragment.

22. The method of claim 20, wherein the ovarian, uterine, prostate, lung, brain, cervical, or fibrosis cell is in a patient.

23. The method of claim 22, wherein the patient is a primate.

24. The method of claim 22, wherein the patient is undergoing a therapeutic regimen to treat metastatic ovarian cancer, uterine cancer, prostate cancer, lung cancer, or cervical cancer.

25. The method of claim 22, wherein the patient is suspected of having metastatic ovarian cancer, uterine cancer, prostate cancer, lung cancer, or cervical cancer.

26. An antibody comprising an amino acid sequence selected from the group of CDR sequences in SEQ ID NO: 7-10.

27. The antibody of claim 26, wherein the antibody is conjugated to an effector component.

28. A pharmaceutical composition comprising a pharmaceutically acceptable excipient and the antibody of claim 26.

29. A method of detecting a cancer or fibrosis cell in a biological sample from a patient, the method comprising contacting the biological sample with an antibody of claim 26.

30. A method of inhibiting proliferation of an ovarian, prostate, lung, or cervical cancer or fibrosis-associated cell, the method comprising the step of contacting the cell with an antibody of claim 26.
